(19) United States
(12) Patent Application Publication
(10) Pub. No.: US 2013/0171222 A1
(43) Pub. Date: Jul. 4, 2013

(54) SILICONE EMULSION

(71) Applicants: Kenji Igarashi, Ibaraki Prefecture (JP); Shinichi Araki, Ibaraki Prefecture (JP)

(72) Inventors: Kenji Igarashi, Ibaraki Prefecture (JP); Shinichi Araki, Ibaraki Prefecture (JP)

(73) Assignee: WACKER CHEMIE AG, Munich (DE)

(21) Appl. No.: 13/821,312

(22) PCT Filed: Aug. 25, 2011
(86) PCT No.: PCT/EP2011/064632

§ 371 Date: Mar. 7, 2013

(30) Foreign Application Priority Data
Sep. 9, 2010 (JP) 2010-202207
May 12, 2011 (JP) 2011-106858

(51) Int. Cl. A61K 8/06; A61Q 5/00; A61K 8/891

(52) U.S. Cl. CPC A61K 8/062; A61K 8/891; A61Q 5/00
USPC 424/401; 424/70.12

(57) ABSTRACT

Stable aqueous silicone emulsion compositions contain a silicone or silicone mixture having a viscosity of 1×10³ to 5×10⁴ mPas, water, and a polyoxyethylated castor oil or hydrogenated castor oil surfactant with a degree of oxyethylation of 150 to 300. The emulsions are stable even in the presence of significant amounts of monoalcohols, and are useful in hair cosmetic formulations.

### CROSS-REFERENCE TO RELATED APPLICATIONS

This application is the U.S. national phase of PCT Appln. No. PCT/EP2011/064632 filed Aug. 25, 2011 which claims priority to Japanese application 2010-202207 filed Sep. 9, 2010, and Japanese application 2011-106858 filed May 12, 2011, the disclosures of which are incorporated in their entirety by reference herein.

### BACKGROUND OF THE INVENTION

### 1. Field of the Invention

The present invention relates to a polyorganosiloxane aqueous emulsion composition used as a material for common cosmetics, and particularly as a material for hair cosmetics.

Polyorganosiloxanes, or silicones, are used as materials for hair cosmetics to impart smooth feeling to the touch, combing properties, and the like. From the viewpoints of adhesion to hair and sustainability of the effects, the polyorganosiloxane preferably has a high viscosity. When the polyorganosiloxane is used for hair cosmetics such as a shampoo, a conditioner, and a rinse, it is common that the polyorganosiloxane is converted into an oil-in-water aqueous emulsion, and then the emulsion is used by mixing with a cosmetic composition. This is because the conversion of the polyorganosiloxane into an emulsion having a specific particle size can develop characteristics of the polyorganosiloxane and facilitate production of cosmetic compositions.

Various particle sizes of such emulsions have been proposed.

For example, Japanese Patent Application Laid-open No. Hei 5-163122 (JP5163122A) describes formulating a polyorganosiloxane aqueous emulsion having an average particle size of 0.2 μm or less. Although the emulsion has excellent stability, the emulsion, with an average particle size of 0.2 μm or less, has a problem in that adhesion of the polyorganosiloxane to hair is inferior, because it tends to be washed out.

On the other hand, Japanese Patent Application Laid-open No. Hei 4-036226 (JP4036226A) proposes that an emulsion having an average particle size of 2 μm or less is mixed with a shampoo or the like, since a shampoo which contains an emulsion with an average particle size of 2 μm or more has decreased foaming properties. When the emulsion has an average particle size of more than 0.3 μm, storage stability of the emulsion is not necessarily sufficient.

To improve adhesion to hair and foaming properties, Japanese Patent Application Laid-open No. Hei 7-188557 (JP7188557A) proposes an organopolysiloxane aqueous emulsion composition having a relatively large particle size of 3 to 100 μm, in which an anionic surfactant of excellent foaming property is used. Japanese Patent Application Laid-open No. Hei 9-316331 (JP9316331A)and Japanese Patent Application Laid-open No. Hei 11-148010 (JP11148010A) propose organopolysiloxane aqueous emulsions having good stability in which a cationic surfactant is used, even though they have a larger particle size. However, there is the problem that emulsions using these ionic surfactants are limited to use in formulations having the same ionicity as in the surfactant when the emulsion is mixed with a composition for hair.

### SUMMARY OF THE INVENTION

It is thus an object of the present invention to provide a stable polyorganosiloxane aqueous emulsion composition which is prepared by emulsifying polyorganosiloxanes having a high viscosity with a nonionic surfactant, and which has a suitable particle size of the dispersed phase in the emulsion to impart smooth feeling to the touch, combing properties, and the like for the use as ingredients in hair cosmetics such as a shampoo, a conditioner, and a rinse.

The inventors of the present inventions have studied earnestly to solve the drawbacks and problems of the prior art, and have found that a polyorganosiloxane aqueous emulsion composition excellent in storage stability having an average particle size of 0.3 μm or more is obtained by emulsifying a polyorganosiloxane having a specific viscosity range with polyoxyethylated castor oil or polyoxyethylated hydrogenated castor oil having a specific molar degree number of oxyethylation.

### DETAILED DESCRIPTION OF THE PREFERRED EMBODIMENTS

More specifically, the present invention is as follows:

[1] an aqueous emulsion composition comprising:

(A) 1 to 90% by weight of a polyorganosiloxane having structural units represented, on average, by the general formula (1), and a viscosity at 25° C. of 1×10³ to 5×10⁴ mPa·s:

R¹ₐSiO_{(4-a)/2} (1)

wherein

R¹ is a substituted or unsubstituted monovalent hydrocarbon group having 1 to 20 carbons or a hydroxyl group;

and a is 1.8 to 2.2;

(B) 0.1 to 20% by weight of a surfactant which is a polyoxyethylated hydrogenated castor oil and/or a polyoxyethylated castor oil, and has a molar number of oxyethylene units of 150 to 300; and

(C) 5 to 98.9% by weight of water.

[2] The aqueous emulsion composition according to [1], wherein the component (A) is a mixture of 5 to 95 parts by weight of one or more kinds of polyorganosiloxane having a viscosity at 25° C. of 2 to 100 mPa·s and 5 to 95 parts by weight of polyorganosiloxane having a viscosity at 25° C. of 1×10⁴ to 1×10⁷ mPa·s, relative to 100 parts by weight of the component (A).

[3] The aqueous emulsion composition according to [1] or [2], wherein R¹ in the general formula (1) is a methyl group.

[4] The aqueous emulsion composition according to [1] to [3], wherein a volume average particle size of an emulsion measured by a Coulter Counter method is 0.3 μm or more.

[5] The aqueous emulsion composition according to [1] to [4], wherein the component (C) is water containing 50% by weight or less of a water-soluble monovalent alcohol.

[6] The aqueous emulsion composition according to [1] to [5] for use in hair cosmetics.

The aqueous polyorganosiloxane emulsion of the present invention is easily mixed with hair cosmetics by using a nonionic surfactant, and it is excellent in storage stability although it has a relatively large particle size of 0.3 μm or more. In conventional aqueous emulsions of polyorganosiloxanes, a thickener such as polyvinyl alcohol and polyacrylamide is added during or after the emulsification process to maintain the emulsion stability. However, in the present invention, the emulsion is stable even without addition of such a thickener. A stabilization component such as a thickener is not preferable since the stabilization component is hard to handle and prevents adhesion of polyorganosiloxane as an active component to hair due to the increased viscosity of the emulsion. Therefore, stability without a thickener is one valuable effect of the present invention. Note that the aqueous emulsion composition of the present invention has a lower viscosity. For example, in the case of formulations where the emulsion has a solids content of 50%, the emulsion has a viscosity as low as about 100 mPa·s, and is convenient for handling as a material for hair cosmetics. Further, the aqueous emulsion of the present invention is stable to water-soluble alcohol, and is stable even in the presence of alcohols such as ethanol, propanol, and isopropanol, which are generally used widely in hair cosmetics.

Hereinafter, the present invention will be described in more detail.

The organopolysiloxane of the component (A) in the present invention is a component for imparting smooth feeling to the touch for hair, combing properties, and the like in the aqueous emulsion. An average structural unit of the siloxane is represented by the general formula (1). In the general formula (1), R¹ is selected from a substituted or unsubstituted monovalent hydrocarbon group having 1 to 20 carbons or a hydroxyl group. Examples of the unsubstituted monovalent hydrocarbon group include alkyl groups such as methyl, ethyl, propyl, butyl, pentyl, hexyl, heptyl, octyl, nonyl, decyl, dodecyl, tetradecyl, hexadecyl, and octadecyl; cycloalkyl groups such as cyclopentyl, and cyclohexyl; aryl groups such as phenyl; and aralkyl groups such as 2-phenylethyl, and 2-phenylpropyl. Substituted monovalent hydrocarbon groups are those obtained by substituting the hydrocarbon group with a functional group. Examples of the functional groups include halogen, epoxy, amino, mercapto, acryloxy, methacryloxy, fatty acid ester, carboxyl, hydroxyl, alkoxy, and polyoxyalkylene. Among them, methyl and/or phenyl are preferable, and 50% by mole or more of methyl groups is particularly preferable.

In the general formula (1), a represents the average number of R¹ to be bonded to the silicon atom of the siloxane, and is 1.8 to 2.2. The molecular structure of the siloxane may have not only a linear chain but also a branched structure, and preferably has a linear chain structure. The organopolysiloxane of the present invention can be prepared by methods known to a person skilled in the art. Specific examples of preferable polyorganosiloxanes, or “silicones” include dimethyl silicones, phenylmethyl silicones, methylhydrogen silicones, alkylaralkyl-modified silicones, fluorine-modified silicones, amino-modified silicones, amino-modified polyether-modified silicones, and amide-modified silicones.

The component (A) in the present invention has a viscosity at 25° C. of 1×10³ to 5×10⁴ mPa·s. When the viscosity of the component (A) at 25° C. is less than 1×10³ mPa·s, adhesion of organopolysiloxane to hair and feeling to the touch are not sufficiently imparted. When the viscosity is more than 5×10⁴ mPa·s, emulsification to obtain a stable emulsion is made difficult. The viscosity at 25° C. is preferably 5×10³ to 5×10⁴ mPa·s, and more preferably 1×10⁴ to 3×10⁴ mPa·s.

The component (A) of the present invention may be a mixture of two or more kinds of polyorganosiloxane having different viscosities as long as the viscosity is within the above-described range. Rather, a mixture, which contains a large amount of polyorganosiloxane component having a high degree of polymerization and high viscosity, is preferable to a polyorganosiloxane having a single molecular weight distribution, as adhesion to hair is increased. In the mixture of polyorganosiloxanes, a low-viscosity polyorganosiloxane component has a viscosity at 25° C. in the range of 2 to 100 mPa·s, and is used in an amount of 5 to 95 parts by weight relative to 100 parts by weight of the component (A). Depending on the viscosity of the high-viscosity component, the low-viscosity component may be a mixture of two or more kinds of polyorganosiloxanes. On the other hand, the high-viscosity polyorganosiloxane component has a viscosity at 25° C. in the range of 1×10⁴ to 1×10⁷ mPa·s, and is used in an amount of 5 to 95 parts by weight relative to 100 parts by weight of the component (A). The component (A) composed of a mixture of these should have a viscosity as a mixture in the range of 1×10³ to 5×10⁴ mPa·s as described above.

When the component (A) is a mixture, it is preferable that the polyorganosiloxane of the low-viscosity component is selected from linear or cyclic polyorganosiloxanes. Examples of cyclic silicones include decamethylcyclopentasiloxane and dodecamethylcyclohexasiloxane.

When the component (A) is a mixture, it is preferable that the polyorganosiloxane of the high-viscosity component is selected from polyorganosiloxanes having a linear or branched structure. Further, as such polyorganosiloxanes having a high viscosity, polyorganosiloxanes having a hydroxyl group at the terminus is suitably used with respect to adhesion to hair.

The component (B) of the present invention is a polyoxyethylated hydrogenated castor oil and/or a polyoxyethylated castor oil, and is a component having the function of a surfactant in emulsification of component (A). In the present invention, the polyoxyethylated hydrogenated castor oil or the polyoxyethylated castor oil needs to have a molar number of polymerized ethylene oxide units of 150 to 300. When the degree of oxyethylation is less than 150, it is not preferable in terms of emulsion stability. When the degree of oxyethylation is more than 300, emulsification and dispersion are not sufficiently accomplished due to increased viscosity. As the HLB values of nonionic surfactant used in emulsification of silicones, various values have been proposed However, suitable HLB values actually used are about 12 to 15. In contrast, the HLB value of the component (B) of the present invention is 17.5 to 19. It is not known to use a nonionic surfactant having such a high HLB value in emulsification of silicones.

The content of component (B) in the present invention is 0.1 to 20% by weight. When the content is less than 0.1% by weight, emulsification is difficult. When the content is more than 20% by weight, an aqueous emulsion composition has high viscosity and is hard to handle. The content is more preferably 3 to 10% by weight.

The water (C) in the present invention is not particularly limited, but it is preferable that ion exchanged water be used. pH is preferably 2 to 12, and more preferably 4 to 10. Although use of calcareous water is discouraged, when used, it is desirable that calcareous water be used with a metal deactivator, or the like. The preferable amount of water isnot particularly limited as an aqueous emulsion, but is preferably 5 to 98.9% by weight, more preferably 10 to 95% by weight, and most preferably 50 to 90% by weight.

The method for producing the emulsion composition of the present invention is not particularly limited, but the emulsion can be produced by known methods. The above components can be mixed and emulsified with an ordinarily mixer suitable for the production of emulsions, such as a homogenizer, a colloid mill, a homomixer, and a high speed rotor-stator mixer, to produce the emulsion. The emulsification can be performed using a method for mixing and stirring all the components (A) to (C) to prepare an oil-in-water emulsion, or a method, in which all or a part of the component (A), a small amount of water (C), and the surfactant (B) are stirred to prepare a water-in-oil emulsion, and the remainder of water is added to the emulsion and stirred to prepare an oil-in-water emulsion. In terms of ease in adjustment of emulsion particle size and stability of emulsion, the method of first preparing a water-in-oil emulsion and then preparing an oil-in-water emulsion is preferable.

It is preferable that the ratio (by weight) of the components (A), (B), and (C) in the present invention be 1 to 90/0.1 to 20/5 to 98.9. When the component (A) has a ratio of more than 90% by weight, it is hard to obtain an aqueous emulsion composition. Lower ratios of component (C) increases the amount of active component. However, when the ratio is less than 5% by weight, it is hard to obtain an oil-in-water aqueous emulsion composition. It is preferable that the ratio (by weight) of the components (A), (B), and (C) be 40 to 70/3 to 10/20 to 57.

The average particle size of the organopolysiloxane aqueous emulsion composition in the present invention is 0.3 μm or more as a volume average particle size. Such a particle size can be measured by the Coulter Counter method which is known by persons skilled in the art. When the volume average particle size is less than 0.3 μm, the adhesion to hair deteriorates. In addition, the viscosity of aqueous emulsion composition increases and handling also is poor. The increased volume average particle size improves adhesion to hair, but deteriorates stability of the aqueous emulsion composition. The largest volume average particle size is about 10 μm. The volume average particle size of emulsion of the present invention is preferably 0.4 to 10 μm, and more preferably 0.5 to 5 μm.

The aqueous emulsion of the present invention is effective in stability to water-soluble alcohols. In general, the water-soluble alcohols have an effect of amphipathicity to aqueous and oil phases. Therefore, it is known that when the emulsion contains the water-soluble alcohols, coalescence of emulsion particles is promoted, reducing the stability of emulsion. Such water-soluble alcohols are monovalent alcohols having 1 to 3 carbons. The aqueous emulsion of the present invention is stable even if water-soluble alcohols are added. For this reason, the emulsion can be widely used in various applications, and particularly in a hair cosmetic formulation containing the water-soluble alcohols. It may be desirable that hair cosmetics contain water-soluble alcohols such as ethanol, propanol, and isopropanol to assist dissolution of other formulating components and adjust viscosity. Therefore, even in such a formulation, the aqueous emulsion of the present invention can be suitably used.

Utilizing the stability of the aqueous emulsion of the present invention even in a system in which water-soluble alcohols are added, the aqueous emulsion can be used as a composition containing the monovalent water-soluble alcohol in advance. In this case, the amount of water-soluble alcohol is such that the content of water-soluble alcohol in the aqueous phase of the component (C) is 50% by weight or less, and preferably 35% by weight or less.

The aqueous emulsion of the present invention may contain other components acceptable as a formulating component of hair cosmetics without impairing the object of the present invention. Examples of the other components include an antiseptic such as quaternary ammonium compounds and phenoxyethanol, various surfactants, thickeners, and perfumes.

The aqueous emulsion of the present invention is suitably used in hair cosmetics, in which silicone is contained as an aqueous emulsion. Further, since the aqueous emulsion has a relatively larger particle size and is stable, it can be used in various applications. Examples of the applications include polish, release agents, fiber, antifoam, and paint applications. The formulation composition in these applications may contain monovalent water-soluble alcohols, if needed.

The aqueous emulsion of the present invention is an aqueous emulsion which is obtained by emulsifying polyorganosiloxane having a high viscosity with a nonionic surfactant, has an emulsion particle size suitable for imparting smooth feeling to the touch for hair, combing properties, and the like, is stable without a stabilizing agent such as a thickener, and is useful for a material for hair cosmetics.

### EXAMPLES

Hereinafter, examples of the present invention will be described in detail, however the present invention is not limited to the following examples. The storage stability test method, particle size measuring method, and viscosity measuring method in the examples are as follows. All values of viscosity were measured at 25° C.

Storage Stability Test Method:

A prepared organopolysiloxane aqueous emulsion was placed into a 50-ml glass bottle, and stored at 40° C. After one month, the presence or absence of creaming, separation, and oil floating on the liquid surface were visually determined. Evaluation standards are as follows.

Pass: there are no creaming, separation, or oil floating on the liquid surface.

Creaming: A concentration distribution of the emulsion is visually observed.

Separation: Creaming is promoted and the aqueous phase is separated, however after shaking, the condition returns into the original emulsion.

Oil floating on the liquid surface: The emulsion is destroyed and an oil content floats.

Particle Size Measuring Method:

The particle size of emulsion was measured with COULTER LS 230 (manufactured by Beckman Coulter, Inc.) The obtained particle size is a volume average particle size.

Viscosity Measuring Method:

The viscosity for oil or emulsion sample of 0.5 ml was measured at 25° C. with Cone/Plate viscometer BROOKFIELD DV-II Pro VISCOMETER CPE 52 (manufactured by Brookfield).

Stability Test in system in which alcohol is added:

10 parts by weight of prepared organopolysiloxane aqueous emulsion was diluted with 90 parts by weight of ethanol aqueous solution having a weight ratio of ethanol to water of 25/65 or 35/55, to prepare an alcohol-added composition. The alcohol-added composition was placed into a 50-ml glass bottle, and stored at 40° C. After one month, the presence or absence of creaming, separation, and oil floating on the liquid surface were visually confirmed. Evaluation standards are as follows.

Pass: there are no creaming, separation, or oil floating on the liquid surface.

Creaming: A concentration distribution of emulsion is visually observed.

Separation: Creaming is promoted and the aqueous phase is separated, however after shaking, the condition returns into the original emulsion.

Oil floating on the liquid surface: The emulsion is destroyed and an oil content floats.

### Example 1

6.2 parts by weight of dimethyl polysiloxane having a viscosity of 3.0×10⁶ mPa·s and dimethylhydroxysilyl groups at both terminii, 19.4 parts by weight of dimethyl polysiloxane having a viscosity of 5.0 mPa·s, and 19.4 parts by weight of dimethyl polysiloxane having a viscosity of 10.0 mPa·s were mixed to prepare 45 parts by weight of organopolysiloxane mixture having a viscosity of 7×10³ mPa·s. This mixture, 5 parts by weight of hydrogenated castor oil having a degree of oxyethylation of 200 mol, and 50 parts by weight of purified water were stirred at 3000 rpm for 20 minutes with ULTRA-TURRAX T 50 basic shaft generator G45G (manufactured by IKA) to prepare an aqueous emulsion. The particle size of the prepared emulsion was measured in accordance with the above particle size measuring method, and storage stability was evaluated in accordance with the above storage stability test method. The evaluation results are shown in Table 1.

### Example 2

An aqueous emulsion was prepared in the same manner as in Example 1 except that 45 parts by weight of an organopolysiloxane mixture having a viscosity of 1.7×10⁴ mPa·s prepared by mixing 22.5 parts by weight of dimethyl polysiloxane having a viscosity of 3.0×10⁵ mPa·s and 22.5 parts by weight of dimethyl polysiloxane having a viscosity of 10 mPa·s, and 5 parts by weight of polyoxyethylated castor oil having a degree of oxyethylene of 200 mol were used. The particle size of the prepared emulsion was measured in accordance with the above particle size measuring method, and storage stability was evaluated in accordance with the above storage stability test method. The evaluation results are shown in Table 1.

### Example 3

An aqueous emulsion was prepared in the same manner as in Example 1 except that 22.5 parts by weight of dimethyl polysiloxane having a viscosity of 3.0×10⁵ mPa·s and 22.5 parts by weight of dimethyl polysiloxane having a viscosity of 10 mPa·s were mixed to prepare 45 parts by weight of organopolysiloxane mixture having a viscosity of 1.7×10⁴ mPa·s. The particle size of the prepared emulsion was measured in accordance with the above particle size measuring method, and storage stability was evaluated in accordance with the above storage stability test method. The evaluation results are shown in Table 1. Further, the stability test of the prepared emulsion in the system in which alcohol is added was performed. The evaluation results are shown in Table 3.

### Example 4

An aqueous emulsion was prepared in the same manner as in Example 1 except that 22.5 parts by weight of dimethyl polysiloxane having a viscosity of 3.0×10⁵ mPa·s and 22.5 parts by weight of dimethyl polysiloxane having a viscosity of 10 mPa·s were mixed to prepare 45 parts by weight of organopolysiloxane mixture having a viscosity of 1.7×10⁴ mPa·s and the shear rate of the mixer was decreased by ½. The particle size of the prepared emulsion was measured in accordance with the above particle size measuring method, and the storage stability was evaluated in accordance with the above storage stability test method. The evaluation results are shown in Table 1.

### Example 5

An aqueous emulsion was prepared in the same manner as in Example 1 except that 33.8 parts by weight of dimethyl polysiloxane having a viscosity of 6.0×10⁴ mPa·s and 11.2 parts by weight of dimethyl polysiloxane having a viscosity of 10 mPa·s were mixed to prepare 45 parts by weight of organopolysiloxane mixture having a viscosity of 1.7×10⁴mPa·s. The particle size of the prepared emulsion was measured in accordance with the above particle size measuring method, and storage stability was evaluated in accordance with the above storage stability test method. The evaluation results are shown in Table 1.

### Example 6

An aqueous emulsion was prepared in the same manner as in Example 1 except that 45 parts by weight of dimethyl polysiloxane having a viscosity of 1.0×10⁴ mPa·s was used instead of the organopolysiloxane mixture. The particle size of the prepared emulsion was measured in accordance with the above particle size measuring method, and storage stability was evaluated in accordance with the above storage stability test method. The evaluation results are shown in Table 1.

### Comparative Example 1

An aqueous emulsion was prepared in the same manner as in Example 2 except that 5 parts by weight of polyoxyethylene hydrogenated castor oil having a degree of oxyethylation of 100 mol was used. The particle size of the prepared emulsion was measured in accordance with the above particle size measuring method, and storage stability was evaluated in accordance with the above storage stability test method. The evaluation results are shown in Table 2. Further, the stability test of the prepared emulsion in the system in which alcohol was added was performed. The evaluation results are shown in Table 3.

### Comparative Example 2

An aqueous emulsion was prepared in the same manner as in Example 2 except that 5 parts by weight of polyoxyethylene tridecyl ether having a degree of oxyalkylation of 10 mol was used. The particle size of the prepared emulsion was measured in accordance with the above particle size measuring method, and storage stability was evaluated in accordance with the above storage stability test method. The evaluation results are shown in Table 2. Further, the stability test of the prepared emulsion in the system in which alcohol was added was performed. The evaluation results are shown in Table 3.

### Comparative Example 3

An aqueous emulsion was prepared in the same manner as in Example 1 except that the shear rate of the mixer was decreased by ⅓. The particle size of the prepared emulsion was measured in accordance with the above particle size measuring method, and storage stability was evaluated in accordance with the above storage stability test method. The evaluation results are shown in Table 2.

TABLE 1

| | | Example | Example | Example | Example | Example | Example |
|---|---|---|---|---|---|---|---|
| Component | | 1 | 2 | 3 | 4 | 5 | 6 |
| (A) | Polydimethyl-siloxane | 6.2 | | | | | |
| | Viscosity 3.0 × 10⁶ | | | | | | |
| | mPa · s | | | | | | |
| | Polydimethyl-siloxane | | 22.5 | 22.5 | 22.5 | | |
| | Viscosity 3.0 × 10⁵ | | | | | | |
| | mPa · s | | | | | | |
| | Polydimethyl-siloxane | | | | | 33.8 | |
| | Viscosity 6.0 × 10⁴ | | | | | | |
| | mPa · s | | | | | | |
| | Polydimethyl-siloxane | | | | | | 45 |
| | Viscosity 1.0 × 10⁴ | | | | | | |
| | mPa · s | | | | | | |
| | Polydimethyl-siloxane | 19.4 | 22.5 | 22.5 | 22.5 | 11.2 | |
| | Viscosity 10 mPa · s | | | | | | |
| | Polydimethyl-siloxane | 19.4 | | | | | |
| | Viscosity 5.0 mPa · s | | | | | | |
| (B) | Polyoxyethylated | | 5 | | | | |
| | Castor Oil | | | | | | |
| | 200 mol EO | | | | | | |
| | Polyoxyethylated | 5 | | 5 | 5 | 5 | 5 |
| | Hydrogenated Castor Oil | | | | | | |
| | 200 mol EO | | | | | | |
| | Polyoxyethylated | | | | | | |
| | Hydrogenated Castor Oil | | | | | | |
| | 100 mol EO | | | | | | |
| | Polyoxyethylated | | | | | | |
| | Tridecyl Ether | | | | | | |
| | 10 mol EO | | | | | | |
| (C) | Purified Water | 50 | 50 | 50 | 50 | 50 | 50 |

| | | | | | | |
|---|---|---|---|---|---|---|
| Total | 100 | 100 | 100 | 100 | 100 | 100 |
| Emulsion Viscosity (mPa · s) | 100 | 100 | 100 | 85 | 100 | 100 |
| Volume Average Particle Size | 1.0 μ | 1.0 μ | 1.0 μ | 5.0 μ | 1.0 μ | 1.0 μ |
| Stability At 40° C. For One Month | Pass | Pass | Pass | Pass | Pass | Pass |
| | | | | (Sign Of | | |
| | | | | Separation) | | |

TABLE 2

| | | Compar- | Compar- | Compar- |
|---|---|---|---|---|
| Component | | ative | ative | ative |
| | | Example 1 | Example 2 | Example 3 |
| (A) | Polydimethylsiloxane | | | 6.2 |
| | Viscosity 3.0 × 106 mPa · s | | | |
| | Polydimethylsiloxane | 22.5 | 22.5 | |
| | Viscosity 3.0 × 105 mPa · s | | | |
| | Polydimethylsiloxane | | | |
| | Viscosity 6.0 × 104 mPa · s | | | |
| | Polydimethylsiloxane | | | |
| | Viscosity 1.0 × 104 mPa · s | | | |
| | Polydimethylsiloxane | 22.5 | 22.5 | 19.4 |
| | Viscosity 10 mPa · s | | | |
| | Polydimethylsiloxane | | | 19.4 |
| | Viscosity 5.0 mPa · s | | | |
| (B) | Polyoxyethylated Castor | | | |
| | Oil | | | |
| | 200 mol EO | | | |
| | Polyoxyethylated | | | 5 |
| | Hydrogenated Castor Oil | | | |
| | 200 mol EO | | | |
| | Polyoxyethylated | 5 | | |
| | Hydrogenated Castor Oil | | | |
| | 100 mol EO | | | |
| | Polyoxyethylated Tridecyl | | 5 | |
| | Ether | | | |
| | 10 mol EO | | | |
| (C) | Purified Water | 50 | 50 | 50 |

| | | | |
|---|---|---|---|
| Total | 100 | 100 | 100 |
| Emulsion Viscosity (mPa · s) | 80 | 80 | 65 |
| Volume Average Particle Size | 1.0 μ | 1.0 μ | 12.0 μ |
| Stability At 40° C. For One Month | Separated | Separated | Separated |
| | In One | In One | In One |
| | Week | Week | Week |

TABLE 3

| | | Example | Example | Comparative | Comparative | Comparative | Comparative |
|---|---|---|---|---|---|---|---|
| Emulsion | | 3 | 3 | Example 1 | Example 1 | Example 2 | Example 2 |
| Composition | Emulsion | 10 | 10 | 10 | 10 | 10 | 10 |
| in | Water | 65 | 55 | 65 | 55 | 65 | 55 |
| Alcohol-added | Ethanol | 25 | 35 | 25 | 35 | 25 | 35 |
| System | | | | | | | |

| | | | | | | |
|---|---|---|---|---|---|---|
| Stability At 40° C. For | Pass | Pass | Oil | Oil | Oil | Oil |
| One Month | | | Floating | Floating | Floating | Floating |
| | | | After One | After One | After One | After One |
| | | | Week | Week | Day | Day |

1.-6. (canceled)

7. An aqueous emulsion composition, comprising:
(A) 1 to 90% by weight of a polyorganosiloxane or a mixture of two or more polyorganosiloxanes having structural units on average represented by formula (1), and a viscosity at 25° C. of 1×10³ to 5×10⁴ mPa·s:
R¹ₐSiO_{(4-a)/2} (1)
wherein
R¹ is a substituted or unsubstituted monovalent hydrocarbon group having 1 to 20 carbons or a hydroxyl group; and
a is 1.8 to 2.2;
(B) 0.1 to 20% by weight of a surfactant which is a polyoxyethylated hydrogenated castor oil and/or polyoxyethylated castor oil, having a degree of polyoxyethylation of 150 to 300; and
(C) 5 to 98.9% by weight of water.

8. The aqueous emulsion composition of claim 7, wherein R¹ in formula (1) is a methyl group.

9. The aqueous emulsion composition of claim 7, wherein the volume average particle size in the emulsion is 0.3 μm or more.

10. The aqueous emulsion composition of claim 8, wherein the volume average particle size in the emulsion is 0.3 μm or more.

11. A hair cosmetic composition, comprising at least one aqueous emulsion composition of claim 7.

12. A hair cosmetic composition, comprising at least one aqueous emulsion composition of claim 8.

13. A hair cosmetic composition, comprising at least one aqueous emulsion composition of claim 9.

14. The aqueous emulsion composition of claim 7, further comprising a C₁-C₃ monoalcohol.

15. The hair cosmetic composition of claim 11, further comprising a C₁-C₃ monoalcohol.
